(19) United States
(12) Patent Application Publication
(10) Pub. No.: US 2026/0327266 A1
(43) Pub. Date: Oct. 22, 2026

(54) RIPPLED ANTIPARALLEL CROSS-BETA DIMERS AND RELATED MATERIALS, COMPOSITIONS AND METHODS

(71) Applicant: The Regents of the University of California, Oakland, CA (US)

(72) Inventors: Jevgenij RASKATOV, Santa Cruz, CA (US); Amaruka Hazari, Santa Cruz, CA (US); Maria Sajimon, Santa Cruz, CA (US)

(21) Appl. No.: 19/312,149

(22) PCT Filed: Mar. 1, 2024
(86) PCT No.: PCT/US2024/018218

§ 371 Date: Aug. 28, 2025

Related U.S. Application Data
(60) Provisional application No. 63/449,903, filed on Mar. 3, 2023.

(51) Int. Cl. C07K 14/00; C07K 1/14; C07K 19/00

(52) U.S. Cl. CPC C07K 14/001; C07K 1/14; C07K 19/00

(57) ABSTRACT

Provided are rippled antiparallel cross-β dimers. In some embodiments, the dimers comprise (L,L,L,L,L,L)-(MVGGVV)ₖ and (D,D,D,D,D,D)-(mvggvv)ₖ, wherein k is an integer of 1 or greater. In other embodiments, the dimers comprise (L,L,L,L,L,L,L)-(KLVFFAE)ₖ and (D,D,D,D,D,D,D)-(klvffae)ₖ, wherein k is an integer of 1 or greater. In yet other embodiments, the dimers comprise (L,L,L,L,L)-(AILSS)ₖ and (D,D,D,D,D)-(ailss)ₖ, wherein k is an integer of 1 or greater. Also provided are rippled β-sheet fibrils comprising a plurality of the rippled antiparallel cross-β dimers of the present disclosure. Materials comprising the rippled antiparallel cross-β dimers and rippled β-sheet fibrils of the present disclosure are also provided, as are compositions comprising such materials. Also provided are methods of making the rippled antiparallel cross-β dimers and rippled β-sheet fibrils of the present disclosure.

### CROSS-REFERENCE TO RELATED APPLICATIONS

This application claims the benefit of U.S. Provisional Patent Application No. 63/449,903, filed Mar. 3, 2023, which application is incorporated herein by reference in its entirety.

### STATEMENT OF GOVERNMENT SUPPORT

This invention was made with Government support under Grant No. 1R01AG074954-01, awarded by the National Institutes of Health. The Government has certain rights in the invention.

### SUMMARY

Provided are rippled antiparallel cross-β dimers. In some embodiments, the dimers comprise (L,L,L,L,L,L)-(MVGGVV)ₖ and (D,D,D,D,D,D)-(mvggvv)ₖ, wherein k is an integer of 1 or greater. In other embodiments, the dimers comprise (L,L,L,L,L,L,L)-(KLVFFAE)ₖ and (D,D,D,D,D,D,D)-(klvffae)ₖ, wherein k is an integer of 1 or greater. In yet other embodiments, the dimers comprise (L,L,L,L,L)-(AILSS)ₖ and (D,D,D,D,D)-(ailss)ₖ, wherein k is an integer of 1 or greater. Also provided are rippled β-sheet fibrils comprising a plurality of the rippled antiparallel cross-β dimers of the present disclosure. Materials comprising the rippled antiparallel cross-β dimers and rippled β-sheet fibrils of the present disclosure are also provided, as are compositions comprising such materials. Also provided are methods of making the rippled antiparallel cross-β dimers and rippled β-sheet fibrils of the present disclosure.

### BRIEF DESCRIPTION OF THE FIGURES

FIG. 1: Fully extended rippled β-sheet layers with FYYF:fyyf.

FIG. 2: Rippled sheet with C-terminus of Aβ40 MVGGVV.

FIG. 3: Rippled sheet with central Aβ40 cluster KLVFFAE.

FIG. 4: The system forms fibers under suitable conditions.

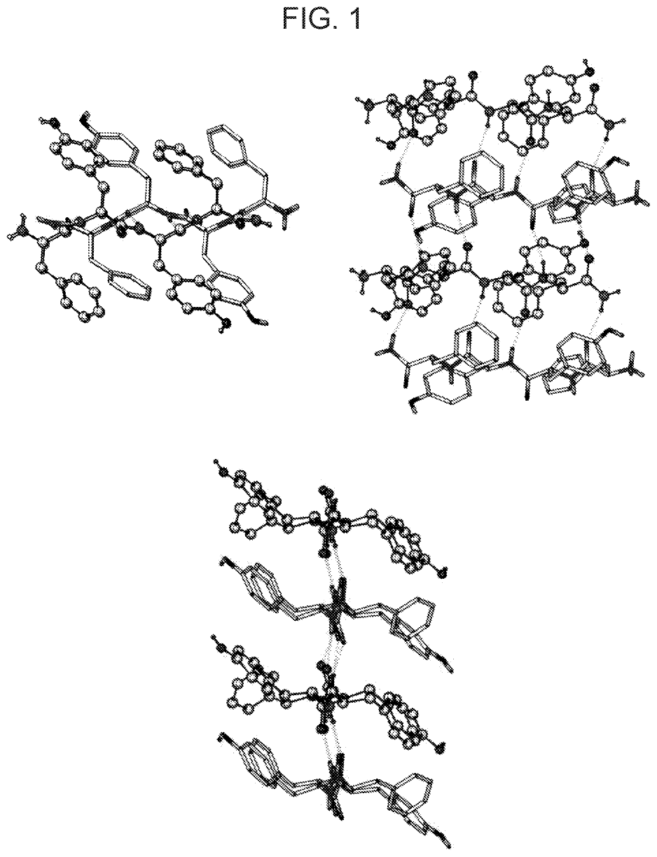

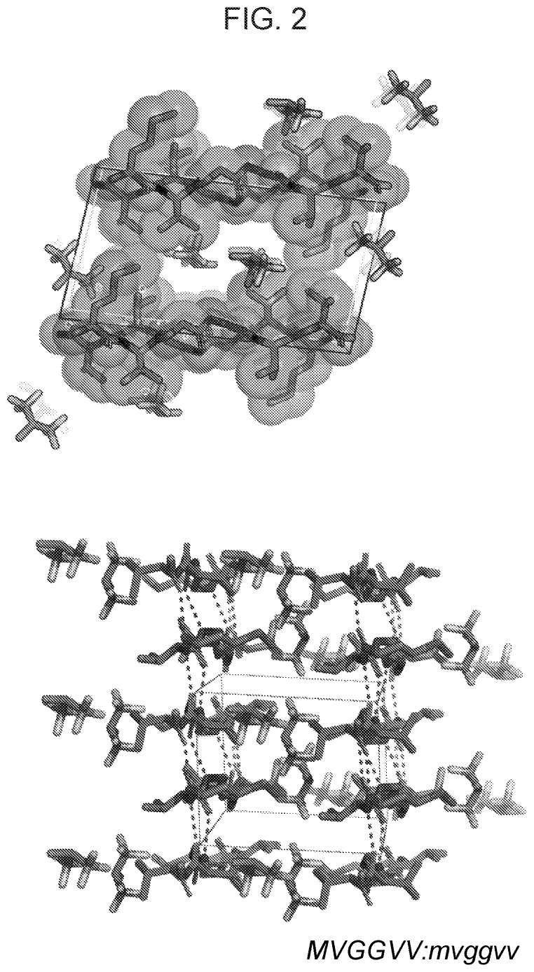

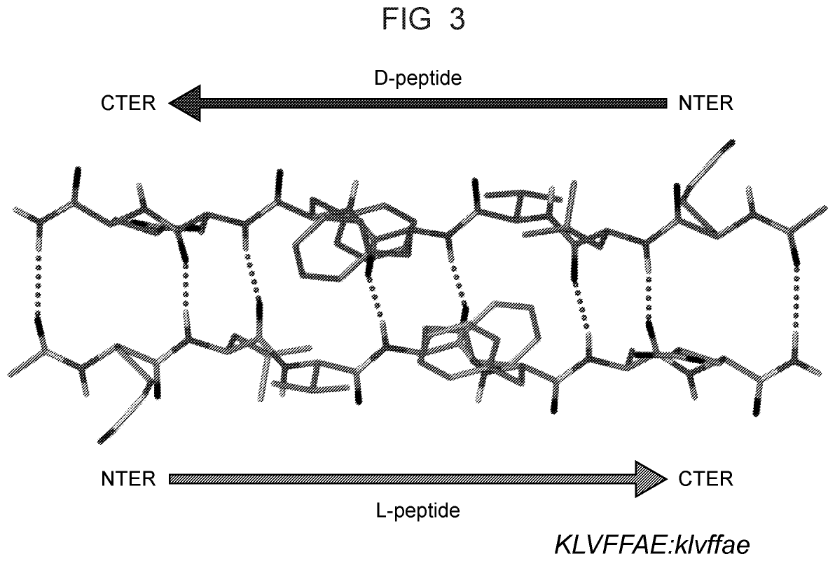

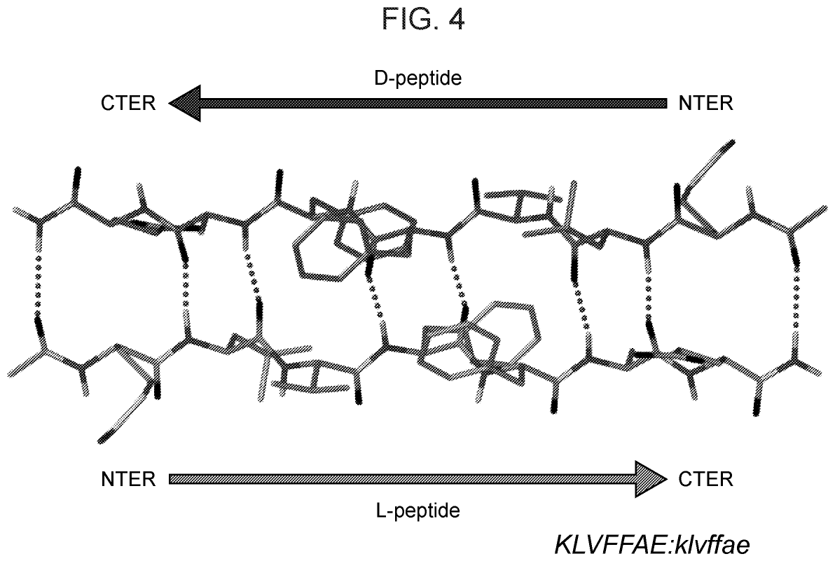

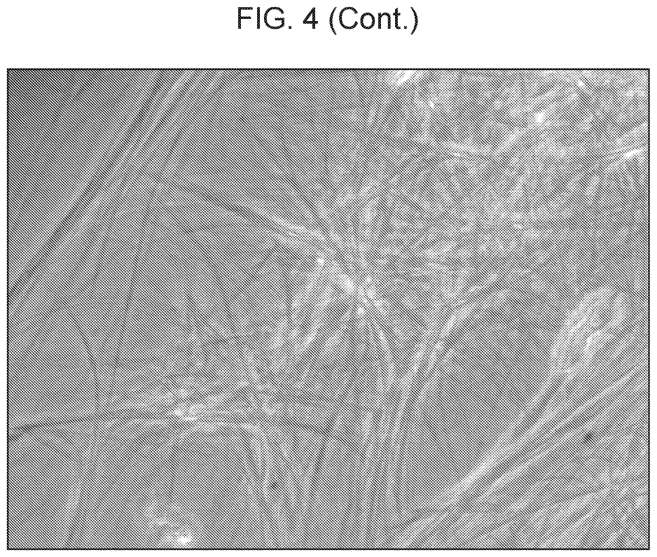

### DETAILED DESCRIPTION

Before the dimers, fibrils and methods of the present disclosure are described in greater detail, it is to be understood that the dimers, fibrils and methods are not limited to particular embodiments described, as such may, of course, vary. It is also to be understood that the terminology used herein is for the purpose of describing particular embodiments only, and is not intended to be limiting, since the scope of the dimers, fibrils and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the dimers, fibrils and methods. The upper and lower limits of these smaller ranges may independently be included in the smaller ranges and are also encompassed within the dimers, fibrils and methods, subject to any specifically excluded limit in the stated range. Where the stated range includes one or both of the limits, ranges excluding either or both of those included limits are also included in the dimers, fibrils and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the dimers, fibrils and methods belong. Although any dimers, fibrils and methods similar or equivalent to those described herein can also be used in the practice or testing of the dimers, fibrils and methods, representative illustrative dimers, fibrils and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present dimers, fibrils and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the dimers, fibrils and methods, which are, for clarity, described in the context of separate embodiments, may also be provided in combination in a single embodiment. Conversely, various features of the dimers, fibrils and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present dimers, fibrils and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Rippled Antiparallel Cross-β Dimers, Rippled β-Sheet Fibrils and Related Materials and Compositions

Aspects of the present disclosure include rippled antiparallel cross-β dimers. The rippled antiparallel cross-β dimers comprise a polymer of two or more L-amino acids dimerized with a polymer of two or more D-amino acids. The term “amino acid” generally refers to any monomer unit that comprises a substituted or unsubstituted amino group, a substituted or unsubstituted carboxy group, and one or more side chains or groups, or analogs of any of these groups. Exemplary side chains include, e.g., thiol, seleno, sulfonyl, alkyl, aryl, acyl, keto, azido, hydroxyl, hydrazine, cyano, halo, hydrazide, alkenyl, alkynl, ether, borate, boronate, phospho, phosphono, phosphine, heterocyclic, enone, imine, aldehyde, ester, thioacid, hydroxylamine, or any combination of these groups. Other representative amino acids include, but are not limited to, amino acids comprising photoactivatable cross-linkers, metal binding amino acids, spin-labeled amino acids, fluorescent amino acids, metal-containing amino acids, amino acids with novel functional groups, amino acids that covalently or noncovalently interact with other molecules, photocaged and/or photoisomerizable amino acids, radioactive amino acids, amino acids comprising biotin or a biotin analog, glycosylated amino acids, other carbohydrate modified amino acids, amino acids comprising polyethylene glycol or polyether, heavy atom substituted amino acids, chemically cleavable and/or photocleavable amino acids, carbon-linked sugar-containing amino acids, redox-active amino acids, amino thioacid containing amino acids, and amino acids comprising one or more toxic moieties.

The term “amino acid” includes, but is not limited to, naturally-occurring α-amino acids and their stereoisomers. “Stereoisomers” of amino acids refer to mirror image isomers of the amino acids, such as L-amino acids or D-amino acids. For example, a stereoisomer of a naturally-occurring amino acid refers to the mirror image isomer of the naturally-occurring amino acid (i.e., the D-amino acid).

Naturally-occurring α-amino acids are those encoded by the genetic code as well as those amino acids that are later modified (e.g., hydroxyproline, γ-carboxyglutamate, and O-phosphoserine). Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Amino acids may be referred to herein by either their commonly known three letter symbols or by the one-letter symbols recommended by the IUPAC-IUB Commission on Biochemical Nomenclature. For example, an L-amino acid may be represented herein by its commonly known three letter symbol (e.g., L-Met for L-methionine) or by an upper-case one-letter amino acid symbol (e.g., M for L-methionine). A D-amino acid may be represented herein by its commonly known three letter symbol (e.g., D-Met for D-methionine) or by a lower-case one-letter amino acid symbol (e.g., m for D-methionine).

The terms “polypeptide,” “peptide,” “protein”, and “polymer of amino acids,” used interchangeably herein, refer to a polymeric form of amino acids of any length (e.g., connected one to the other by peptide bonds between the alpha-amino and carboxy groups of adjacent residues), which can include genetically coded and non-genetically coded amino acids, chemically or biochemically modified or derivatized amino acids, and polypeptides having modified peptide backbones.

In certain embodiments, provided are rippled antiparallel cross-β dimers comprising (L,L,L,L,L,L)-(MVGGVV)ₖ dimerized with (D,D,D,D,D,D)-(mvggvv)ₖ, wherein k is an integer of 1 or greater.

According to some embodiments, provided are rippled antiparallel cross-β dimer comprising (L,L,L,L,L,L,L)-(KLVFFAE)ₖ dimerized with (D,D,D,D,D,D,D)-(klvffae)ₖ, wherein k is an integer of 1 or greater.

In other embodiments, provided are rippled antiparallel cross-β dimer comprising (L,L,L,L,L)-(AILSS)ₖ dimerized with (D,D,D,D,D)-(ailss)ₖ, wherein k is an integer of 1 or greater.

“L” refers to levorotatory, and “D” refers to dextrorotatory. According to some embodiments, k is an integer of from 1 to 1000, such as from 1 to 750, from 1 to 500, from 1 to 250, from 1 to 100, from 1 to 75, from 1 to 50, from 1 to 40, from 1 to 30, from 1 to 20, or from 1 to 10.

Also provided by the present disclosure are rippled β-sheet fibrils. The rippled β-sheet fibrils comprise a plurality of the rippled antiparallel cross-β dimers of the present disclosure.

Aspects of the present disclosure further include materials comprising a plurality of the rippled antiparallel cross-β dimers of the present disclosure. According to some embodiments, provided are materials comprising the rippled β-sheet fibrils of the present disclosure. In certain embodiments, such materials comprise the plurality of rippled antiparallel cross-β dimers held together by a combination of interdimer hydrogen bonds, ionic interactions, van der Waals interactions, or any combination thereof. For example, the materials may comprise the plurality of rippled antiparallel cross-β dimers held together by a combination of interdimer hydrogen bonds, ionic interactions, and van der Waals interactions.

Also provided are compositions comprising the materials of the present disclosure. In certain embodiments, such a composition includes a material of the present disclosure present in a liquid medium, e.g., an aqueous liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino) ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a protease inhibitor, glycerol, a chelating agent, and the like may be present in such compositions.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the materials against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included, e.g., in an amount of about 10 mM to 500 nM.

In certain embodiments, a composition of the present disclosure comprises the material and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the composition, e.g., at concentrations ranging from about 0.001 to about 2% weight/volume (w/v).

### Methods

Also provided by the present disclosure are methods of making and using the rippled antiparallel cross-β dimers, rippled β-sheet fibrils, materials and compositions of the present disclosure.

In certain embodiments, provided are methods comprising producing a polypeptide comprising, consisting essentially of, or consisting of, (L,L,L,L,L,L)-(MVGGVV)ₖ, and producing a polypeptide comprising, consisting essentially of, or consisting of, (D,D,D,D,D,D)-(mvggvv)ₖ, wherein k is an integer of 1 or greater. According to some embodiments, provided are methods comprising producing a polypeptide comprising, consisting essentially of, or consisting of, (L,L,L,L,L,L,L)-(KLVFFAE)ₖ, and producing a polypeptide comprising, consisting essentially of, or consisting of, (D,D,D,D,D,D,D)-(klvffae)ₖ, wherein k is an integer of 1 or greater. In certain embodiments, provided are methods comprising producing a polypeptide comprising, consisting essentially of, or consisting of, (L,L,L,L,L)-(AILSS)ₖ, and producing a polypeptide comprising, consisting essentially of, or consisting of, (D,D,D,D,D)-(ailss)ₖ, wherein k is an integer of 1 or greater.

According to some embodiments, the polypeptides are produced by chemical synthesis. A non-limiting example of a chemical synthesis includes solid-phase polypeptide synthesis. Solid-phase peptide synthesis (SPPS) involves the successive addition of protected amino acid derivatives to a growing peptide chain immobilized on a solid phase, including deprotection and washing steps to remove unreacted groups and also side products. Any number of solid supports may be employed, including resins such as polystyrene and polyamide based resins. The peptides may be covalently bound to a solid support, typically at their C-terminal end through linkers such as acid labile and photolabile linkers. In some embodiments the linker is an acid labile linker. In other embodiments, the linker is a trityl linker such as a 2-chlorotrityl linker. Peptide synthesis is typically performed by coupling a protected amino acid to the N-terminal end of the bound sample. The protected amino acid may contain N-terminal protecting groups such as a Boc (tert-butyloxycarbonyl) or Fmoc (9-fluorenylmethyloxycarbonyl) group as well as side chain protecting groups. According to some embodiments, when solid-phase polypeptide synthesis is performed to produce the polypeptides, the solid-phase synthesis is Fmoc-based solid-phase synthesis.

A non-limiting example approach for producing polypeptide monomers of the cross-β dimers of the present disclosure will now be described. The olypeptides may be synthesized using preloaded, Fmoc-[amino acid] 4-alkoxybenzyl alcohol Wang resin: Fmoc-L-[amino acid]-Wang (Sigma) or Fmoc-D-[amino acid]-Wang (Fisher). Syntheses may be performed manually at 0.2 mM scale relative to resin loading. An orbital shaker may be used for mixing in both the deprotection and coupling steps. The resin may be swelled in 3 mL of dimethylformamide (DMF) in a filter tube, housing 250 mg Fmoc-[amino acid] Wang resin (0.796 mmol/g loading) for 20 min. For Fmoc-deprotection, 30% piperidine (Spectrum) in DMF may be added to the resin, and allowed to shake on an orbital shaker for 20 min. The deprotection solution may be rinsed with DMF (3×) and dichloromethane (DCM, 2×) and the deprotection step may be repeated. Coupling reagents used may be 4 eq. N,N-diisopropylethylamine (Fisher), 3 eq. N,N,N′,N′-tetramethyl-O-(1H-benzotriazol-1-yl)uronium hexafluorophosphate (Fisher) and 3 eq. hydroxybenzotriazole hydrate (Oakwood Products). For amino acid coupling, 3 eq. of either Fmoc-L-[amino acid]-OH (Fisher) or Fmoc-D-[amino acid]-OH (ChemPep) with coupling reagents listed above may be dissolved in 3 mL DMF and added to the reaction vessel, and allowed to shake for 30 min. The coupling step may be repeated for each amino acid addition to improve yield. The aforementioned steps are repeated to produce the resin-bound polypeptides, NH₂-L-polypeptide-COOH and NH₂-D-polypeptide-COOH. The polypeptides may be cleaved and deprotected with a mixture consisting of trifluoroacetic acid (10 mL, Fisher), tri-isopropylsilane (1 mL, Fisher), and liquefied phenol (0.5 mL, Sigma). The polypeptide identities may be confirmed with mass spectrometry.

The above-described methods may further comprise purifying the produced polypeptides. Any suitable approach for purifying the polypeptides may be employed. In certain embodiments, the polypeptides are purified by chromatography, a non-limiting example of which is High Performance Liquid Chromatography (HPLC). An example HPLC-based approach suitable for purifying the polypeptides is described in Warner et al. (2017) JoVE, 2017, e55482. In some instances, polypeptides may be purified by reverse-phase high-performance liquid chromatography (HPLC) with PLRP-S columns (Agilent), yielding polypeptides with purities exceeding 95%. HPLC may be conducted under basic conditions (0.1% NH₄OH), to reduce aggregation and/or precipitation. Samples may be lyophilized and stored as solid powders at −40 °C.

The above-described methods may further comprise combining the produced polypeptides into a racemic mixture. In certain embodiments, the combining is under conditions suitable for formation of the rippled antiparallel cross-β dimers of the present disclosure. For example, according to some embodiments, provided are methods that comprise combining the polypeptides in a mixture under conditions in which rippled antiparallel cross-β dimers are formed. According to some embodiments, the combining is under conditions suitable for formation of a rippled β-sheet fibril of the present disclosure. Approaches for dimerizing polypeptides in a manner suitable for producing the dimers of the present disclosure are known and described in, e.g., Hazari et al. (2022) Chem. Sci. 13:8947-8952; and Raskatov et al. (2021) Acc. Chem. Res. 54(10): 2488-2501; Raskatov, J. (2020) ChemBioChem 21(20): 2868; and International Patent Application Publication No. WO2022266494A1; the disclosures of which are incorporated herein in their entireties for all purposes.

Notwithstanding the appended claims, the present disclosure is also defined by the following embodiments:
1. A rippled antiparallel cross-β dimer comprising (L,L,L,L,L,L)-(MVGGVV)ₖ dimerized with (D,D,D,D,D,D)-(mvggvv)ₖ, wherein k is an integer of 1 or greater.
2. A rippled antiparallel cross-β dimer comprising (L,L,L,L,L,L,L)-(KLVFFAE)ₖ dimerized with (D,D,D,D,D,D,D)-(klvffae)ₖ, wherein k is an integer of 1 or greater.
3. A rippled antiparallel cross-β dimer comprising (L,L,L,L,L)-(AILSS)ₖ dimerized with (D,D,D,D,D)-(ailss)ₖ, wherein k is an integer of 1 or greater.
4. A rippled β-sheet fibril comprising a plurality of the rippled antiparallel cross-β dimers of any one of embodiments 1 to 3.
5. A material comprising a plurality of the rippled antiparallel cross-β dimers of any one of embodiments 1 to 3.
6. A material comprising the rippled β-sheet fibril of embodiment 4.
7. The material of embodiment 5 or embodiment 6, wherein the material comprises the plurality of rippled antiparallel cross-β dimers held together by a combination of interdimer hydrogen bonds, ionic interactions, and van der Waals interactions.
8. A composition comprising the rippled antiparallel cross-β dimers of any one of embodiments 1 to 3, the rippled β-sheet fibril of embodiment 4, or the material of any one of embodiments 5 to 7.
9. The composition of embodiment 8, wherein the dimer or material is present in a liquid medium.
10. The composition of embodiment 9, wherein the liquid medium is an aqueous liquid medium.
11. A method comprising:
   producing a polypeptide comprising, consisting essentially of, or consisting of, (L,L,L,L,L,L)-(MVGGVV)ₖ; and
   producing a polypeptide comprising, consisting essentially of, or consisting of, (D,D,D,D,D,D)-(mvggvv)ₖ,
   wherein k is an integer of 1 or greater.
12. A method comprising:
   producing a polypeptide comprising, consisting essentially of, or consisting of, (L,L,L,L,L,L,L)-(KLVFFAE)ₖ; and
   producing a polypeptide comprising, consisting essentially of, or consisting of, (D,D,D,D,D,D,D)-(klvffae)ₖ,
   wherein k is an integer of 1 or greater.
13. A method comprising:
   producing a polypeptide comprising, consisting essentially of, or consisting of, (L,L,L,L,L)-(AILSS)ₖ; and
   producing a polypeptide comprising, consisting essentially of, or consisting of, (D,D,D,D,D)-(ailss)ₖ,
   wherein k is an integer of 1 or greater.
14. The method according to any one of embodiments 11 to 13, wherein the polypeptides are produced by chemical synthesis.
15. The method according to embodiment 14, wherein the chemical synthesis is by solid-phase polypeptide synthesis.
16. The method according to embodiment 15, wherein the solid-phase polypeptide synthesis is Fmoc-based solid-phase peptide synthesis.
17. The method according to any one of embodiments 11 to 16, further comprising purifying the produced polypeptides.
18. The method according to any one of embodiments 11 to 17, further comprising combining the produced polypeptides into a racemic mixture.
19. A method comprising:
   combining (L,L,L,L,L,L)-(MVGGVV)ₖ and (D,D,D,D,D,D)-(mvggvv)ₖ in a mixture under conditions in which rippled antiparallel cross-β dimers comprising (L,L,L,L,L,L)-(MVGGVV)ₖ and (D,D,D,D,D,D)-(mvggvv)ₖ are formed, wherein k is an integer of 1 or greater.
20. A method comprising:
   combining (L,L,L,L,L,L,L)-(KLVFFAE)ₖ and (D,D,D,D,D,D,D)-(klvffae)ₖ in a mixture under conditions in which rippled antiparallel cross-β dimers comprising (L,L,L,L,L,L,L)-(KLVFFAE)ₖ and (D,D,D,D,D,D,D)-(klvffae)ₖ are formed, wherein k is an integer of 1 or greater.
21. A method comprising:
   combining (L,L,L,L,L)-(AILSS)ₖ and (D,D,D,D,D)-(ailss)ₖ in a mixture under conditions in which rippled antiparallel cross-β dimers comprising (L,L,L,L,L)-(AILSS)ₖ and (D,D,D,D,D)-(ailss)ₖ are formed, wherein k is an integer of 1 or greater.
22. The method according to any one of embodiments 19 to 21, wherein a rippled β-sheet fibril comprising the rippled antiparallel cross-β dimers is formed.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A rippled antiparallel cross-β dimer comprising (L,L,L,L,L,L)-(MVGGVV)ₖ dimerized with (D,D,D,D,D,D)-(mvggvv)ₖ, wherein k is an integer of 1 or greater.

2. A rippled antiparallel cross-β dimer comprising (L,L,L,L,L,L,L)-(KLVFFAE)ₖ dimerized with (D,D,D,D,D,D,D)-(klvffae)ₖ, wherein k is an integer of 1 or greater.

3. A rippled antiparallel cross-β dimer comprising (L,L,L,L,L)-(AILSS)ₖ dimerized with (D,D,D,D,D)-(ailss)ₖ, wherein k is an integer of 1 or greater.

4. A rippled β-sheet fibril comprising a plurality of the rippled antiparallel cross-β dimers of any one of claims 1 to 3.

5. A material comprising a plurality of the rippled antiparallel cross-β dimers of any one of claims 1 to 3.

6. A material comprising the rippled β-sheet fibril of claim 4.

7. The material of claim 5 or claim 6, wherein the material comprises the plurality of rippled antiparallel cross-β dimers held together by a combination of interdimer hydrogen bonds, ionic interactions, and van der Waals interactions.

8. A composition comprising the rippled antiparallel cross-β dimers of any one of claims 1 to 3, the rippled β-sheet fibril of claim 4, or the material of any one of claims 5 to 7.

9. The composition of claim 8, wherein the dimer, fibril or material is present in a liquid medium.

10. The composition of claim 9, wherein the liquid medium is an aqueous liquid medium.

11. A method comprising:
producing a polypeptide comprising, consisting essentially of, or consisting of, (L,L,L,L,L,L)-(MVGGVV)ₖ; and
producing a polypeptide comprising, consisting essentially of, or consisting of, (D,D,D,D,D,D)-(mvggvv)ₖ,
wherein k is an integer of 1 or greater.

12. A method comprising:
producing a polypeptide comprising, consisting essentially of, or consisting of, (L,L,L,L,L,L,L)-(KLVFFAE)ₖ; and
producing a polypeptide comprising, consisting essentially of, or consisting of, (D,D,D,D,D,D,D)-(klvffae)ₖ,
wherein k is an integer of 1 or greater.

13. A method comprising:
producing a polypeptide comprising, consisting essentially of, or consisting of, (L,L,L,L,L)-(AILSS)ₖ; and
producing a polypeptide comprising, consisting essentially of, or consisting of, (D,D,D,D,D)-(ailss)ₖ,
wherein k is an integer of 1 or greater.

14. The method according to any one of claims 11 to 13, wherein the polypeptides are produced by chemical synthesis.

15. The method according to claim 14, wherein the chemical synthesis is by solid-phase polypeptide synthesis.

16. The method according to claim 15, wherein the solid-phase polypeptide synthesis is Fmoc-based solid-phase peptide synthesis.

17. The method according to any one of claims 11 to 16, further comprising purifying the produced polypeptides.

18. The method according to any one of claims 11 to 17, further comprising combining the produced polypeptides into a racemic mixture.

19. A method comprising:
combining (L,L,L,L,L,L)-(MVGGVV)ₖ and (D,D,D,D,D,D)-(mvggvv)ₖ in a mixture under conditions in which rippled antiparallel cross-β dimers comprising (L,L,L,L,L,L)-(MVGGVV)ₖ and (D,D,D,D,D,D)-(mvggvv)ₖ are formed, wherein k is an integer of 1 or greater.

20. A method comprising:
combining (L,L,L,L,L,L,L)-(KLVFFAE)ₖ and (D,D,D,D,D,D,D)-(klvffae)ₖ in a mixture under conditions in which rippled antiparallel cross-β dimers comprising (L,L,L,L,L,L,L)-(KLVFFAE)ₖ and (D,D,D,D,D,D,D)-(klvffae)ₖ are formed, wherein k is an integer of 1 or greater.

21. A method comprising:
combining (L,L,L,L,L)-(AILSS)ₖ and (D,D,D,D,D)-(ailss)ₖ in a mixture under conditions in which rippled antiparallel cross-β dimers comprising (L,L,L,L,L)-(AILSS)ₖ and (D,D,D,D,D)-(ailss)ₖ are formed, wherein k is an integer of 1 or greater.

22. The method according to any one of claims 19 to 21, wherein a rippled β-sheet fibril comprising the rippled antiparallel cross-β dimers is formed.
